(19) United States
(12) Patent Application Publication
(10) Pub. No.: US 2026/0325731 A1
(43) Pub. Date: Oct. 22, 2026

(54) DEVICES, SYSTEMS AND METHODS FOR EXTRACORPOREAL MEMBRANE OXYGENATION

(71) Applicant: PAVmed, Inc., New York, NY (US)

(72) Inventors: Michael Boutillette, Sudbury, MA (US); Gregory Eberl, Concord, MA (US); Lishan Aklog, Purchase, NY (US); Brian deGuzman, Paradise Valley, AZ (US); Jessie Gifford, Sharon, MA (US); Anthony Maiorano, North Attleboro, MA (US); Tim Robinson, Foxborough, MA (US); Deborah Leszczynski, Boston, MA (US)

(21) Appl. No.: 19/119,384

(22) PCT Filed: Oct. 31, 2023
(86) PCT No.: PCT/US23/36387

§ 371 Date: Apr. 8, 2025

Related U.S. Application Data
(60) Provisional application No. 63/381,884, filed on Nov. 1, 2022.

(51) Int. Cl. A61M 1/36; A61M 60/113; A61M 60/38

(52) U.S. Cl. CPC A61M 1/3659; A61M 1/3666; A61M 60/113; A61M 60/38; A61M 2210/125

(57) ABSTRACT

In some examples, a vascular medical device includes a first cannula having a proximal opening and a distal opening, and defining a first lumen, and a second cannula having a proximal opening and a distal opening, and defining a second lumen, the second cannula comprising an expandable distal portion configured to transition between a collapsed state having a first cross-sectional area, to an expanded state having a second cross-sectional area that is greater than the first cross-sectional area.

### CROSS REFERENCE TO RELATED APPLICATIONS

The present application claims the benefit of priority to U.S. Provisional Application No. 63/381,884, filed Nov. 1, 2022, and titled “Devices, Systems and Methods for Extracorporeal Membrane Oxygenation.”

### FIELD OF THE DISCLOSURE

The present disclosure relates to devices, systems, and methods for extracorporeal membrane oxygenation. More particularly, the present disclosure relates to an expandable vascular medical device for use in systems and methods that extracorporeally oxygenate blood.

### BACKGROUND

Extracorporeal membrane oxygenation (ECMO) systems provide life support for individuals who have a severe and life-threatening illness that stops the heart and/or lungs from working properly. For example, an ECMO system is used during life-threatening conditions such as severe lung damage from infection, or shock after a massive heart attack.

ECMO machinery replaces the function of the heart and/or lungs. Typically, patients are supported by an ECMO machine for only a few hours to days, but some may require it for a few weeks, depending on how their condition progresses. An ECMO machine pumps and oxygenates a patient's blood outside the body, allowing the lungs, and in some instances also the heart, to rest. When a patient is connected to an ECMO system, deoxygenated blood is pumped out of the patient's body through tubing to an artificial lung in the machine that adds oxygen and removes carbon dioxide; then the extracorporeally oxygenated blood is warmed to body temperature and pumped back into the body. There are two types of ECMO. Veno-Arterial (“VA”) ECMO systems typically connect to both the patient's veinous vasculature and the patient's arterial vasculature. VA ECMO is traditionally used when there are problems with both the patient's heart and lungs. Veno-Veno (“VV”) ECMO systems typically connect to one or more of the patient's veins, usually near the patient's heart. VV ECMO is traditionally used when there is a problem with only the patient's lungs.

### SUMMARY

There is a need for improvements to the vasculature devices within extracorporeal membrane oxygenation (“ECMO”) systems. The present disclosure is directed towards solutions to address this need, in addition to having other desirable characteristics. Specifically, the present disclosure provides an expandable vascular device that can be implemented as part of ECMO systems to provide easier access to areas of the patients'vasculature and heart.

In accordance with some embodiments, a vascular medical device is provided. The device includes a first cannula that has a proximal opening and a distal opening, where the first cannula defines a first lumen, and a second cannula having a proximal opening and a distal opening, where the second cannula defines a second lumen and the second cannula includes an expandable distal portion configured to transition between a collapsed state that has a first cross-sectional area, to an expanded state that has a second cross-sectional area that is greater than the first cross-sectional area.

In accordance with some embodiments, a method of treatment is provided. The method includes providing a first cannula that has a proximal opening and a distal opening, where the first cannula defines a first lumen, providing a second cannula that has a proximal opening and a distal opening, where the second cannula defines a second lumen and the second cannula includes an expandable distal portion. The method also includes placing the second cannula at a first target position, placing the expandable distal portion of the second cannula at a second target position, and transitioning the expandable distal portion from a collapsed state that has a first cross-sectional area, to an expanded state that has a second cross-sectional area that is greater than the first cross-sectional area.

In accordance with some embodiments, an extracorporeal membrane oxygenator system for extracorporeal oxygenation of blood and removal of carbon dioxide from the blood is provided. The system includes a pump for pumping blood, an oxygenator for exchanging oxygen and carbon dioxide within the pumped blood, and a vascular medical device, where the vascular medical device includes a first cannula that has a proximal opening and a distal opening, where the first cannula define a first lumen, and the vascular medical device includes a second cannula that has a proximal opening and a distal opening, where the second cannula defines a second lumen and the second cannula includes an expandable distal portion configured to transition between a collapsed state that has a first cross-sectional area, to an expanded state that has a second cross-sectional area that is greater than the first cross-sectional area.

### BRIEF DESCRIPTION OF THE FIGURES

These and other characteristics of the present disclosure will be more fully understood by reference to the following detailed description in conjunction with the attached drawings, in which:

FIG. 1 illustrates an exemplary ECMO system utilizing a vascular medical device and placed at a first target position within a patient, in accordance with some embodiments of the present disclosure;

FIG. 2 illustrates exemplary ECMO machinery, in accordance with some embodiments of the present disclosure;

FIG. 3 is an exemplary cross-sectional side view of a vascular medical device, in accordance with some embodiments of the present disclosure;

FIGS. 4A, 4B, 4C, 4D, 4E, and 4F are exemplary cross-sectional end views of vascular medical devices, in accordance with some embodiments of the present disclosure;

FIG. 5A is an exemplary cross-sectional side view of a vascular medical device within a patient's cardiac anatomy at a first target position with an expandable portion in a collapsed state, in accordance with some embodiments of the present disclosure;

FIG. 5B is an exemplary cross-sectional side view of the vascular medical device of FIG. 5A within a patient's cardiac anatomy at a second target position with the expandable portion in a collapsed state, in accordance with some embodiments of the present disclosure;

FIG. 5C is an exemplary cross-sectional side view of the vascular medical device of FIG. 5B within a patient's cardiac anatomy at the second target position with the expandable portion in an expanded state, in accordance with some embodiments of the present disclosure;

FIG. 6A is an exemplary cross-sectional side view of a vascular medical device within a patient's cardiac anatomy at another first target position with an expandable portion in a collapsed state, in accordance with some embodiments of the present disclosure;

FIG. 6B is an exemplary cross-sectional side view of the vascular medical device of FIG. 6A within a patient's cardiac anatomy at another second target position with the expandable portion in a collapsed state, in accordance with some embodiments of the present disclosure;

FIG. 6C is an exemplary cross-sectional side view of the vascular medical device of FIG. 6B within a patient's cardiac anatomy at the second target position with the expandable portion in an expanded state, in accordance with some embodiments of the present disclosure;

FIG. 7 illustrates an exemplary cross sectional side view of a vascular medical device within a patient's cardiac anatomy at an additional second target position.

FIG. 8A illustrates a side view of an exemplary stent in accordance with some embodiments of the present disclosure; and

FIG. 8B illustrates a side view of an exemplary stent in accordance with other embodiments of the present disclosure.

### DETAILED DESCRIPTION

An illustrative embodiment of the present disclosure relates to a vascular medical device for use in extracorporeal membrane oxygenation (“ECMO”) systems. The vascular medical device is configured to provide an expandable portion of a cannula. The expandable portion preferably has minimal initial dimensions and is maneuverable such that it may be manipulated to reach various desired locations within a patient's vasculature and/or cardiac anatomy. For example, the expandable distal portion may be configured for being maneuvered into the patient's Right Ventricle, or through a transseptal puncture into the patient's Left Atrium, or through a transseptal puncture into the patient's Left Atrium continuing into the patient's Left Ventricle, or through a transeptal puncture into the patient's Left Atrium continuing throughout the patient's Left Ventricle and into the patient's Aorta.

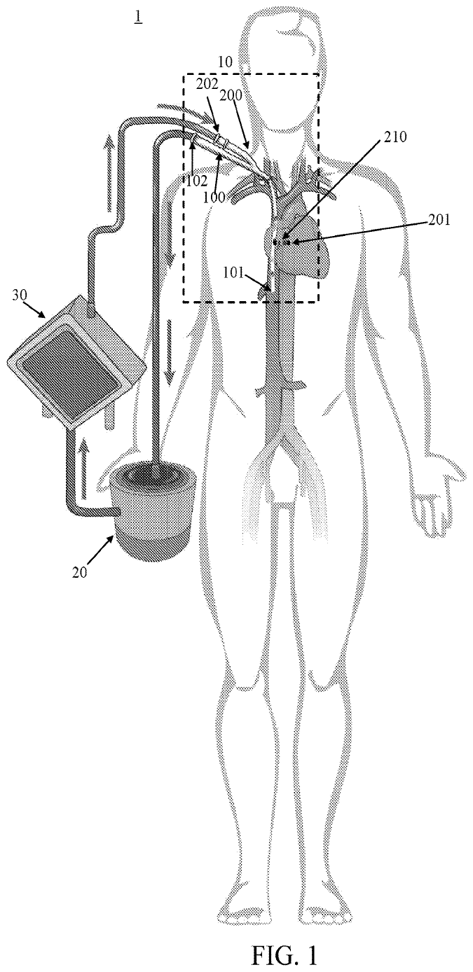

Once the expandable portion is maneuvered to a desired location, it may be expanded to dimensions appropriate for containing fluid flowing at rates sufficient for the medical needs of the patient. For example, with reference to FIG. 1, an ECMO system 1 may include ECMO machinery, such as a pump 20 and an oxygenator 30, connected to at least one vascular medical device 10 having an expandable portion 210. The device 10 may provide one cannula 100 for removing deoxygenated blood from a patient's vasculature and a second cannula 200 for returning oxygenated blood to the patient's vasculature. The expandable portion 210 of the device 10 may be at the distal end 201 of the second cannula 200. The device 10 may be inserted into a patient's vasculature via the Jugular Vein and advanced through the Vena Cava such that the first cannula 100 extends from a distal opening 101 within the patient's Inferior Vena Cava, travels into the patient's Right Atrium, continues out of the patient's heart into the patient's Superior Vena Cava and exits the patient's Jugular Vein. The second cannula 200 may be placed with the expandable distal portion 210 within the patient's vasculature and/or cardiac anatomy, such as, for example, within the Right Atrium with the second cannula 200 continuing out of the patient's heart, into the patient's Superior Vena Cava, and exiting the patient's Jugular Vein. The proximal opening 102 of the first cannula 100 may be connected to, or otherwise in fluid communication with, a pump 20 of the ECMO system 1, which may be connected to, or otherwise in fluid communication with, an oxygenator 30 of the ECMO system 1. The proximal opening of the second cannula 202 may be connected to, or otherwise in fluid communication with, the oxygenator 30. Once the expandable distal portion 210 of the second cannula 200 is placed at a desired location, the patient's deoxygenated blood can be pumped out of the patient's Vena Cava and Right Atrium via the first cannula 100, oxygenated, and returned to the patient's vasculature and/or cardiac anatomy via the second cannula 200. In some embodiments, the expandable distal portion 210 may be self-expanding and allowed to expand once placed in the desired location, while in other embodiments the returning oxygenated blood may be pumped through the second cannula 200 at a pressure great enough to cause the expansion of the distal portion 210 of the second cannula 200. Preferably, the expandable distal portion 210 is configured to have dimensions when expanded that allow the returning oxygenated blood to flow back into the patient's vasculature and/or cardiac anatomy at rates sufficient for supporting and/or maintaining the patient's circulation. In other words, the present disclosure may be used to provide ECMO systems with access to, and ability to return oxygenated blood to, various locations within the patient's vasculature and/or cardiac anatomy.

FIGS. 1 and 3 through 6C, wherein like parts are designated by like reference numerals throughout, illustrate an example embodiment or embodiments of improved vascular medical devices, ECMO systems, and methods of use, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

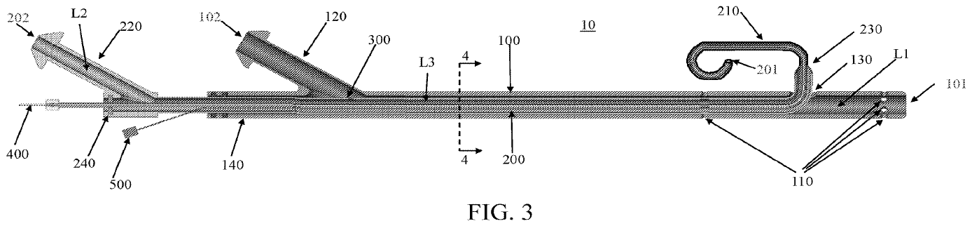

Referring to FIG. 3, an exemplary embodiment, in cross-sectional view, of a vascular medical device is depicted. The vascular medical device 10 includes a first cannula 100 and a second cannula 200 disposed at least partially within the first cannula 100. The first cannula 100 includes a proximal opening 102 and a distal opening 101 and defines a first lumen L1. The second cannula 200 includes a proximal opening 202 and a distal opening 201 and defines a second lumen L2. A portion 210 of the second cannula 200 is expandable. In certain embodiments, the expandable portion 210 is distal to, or disposed outside, other portion(s) of the second cannula. The expandable portion 210 is configured to transition from a collapsed state to an expanded state, where it has a first cross-sectional area in the collapsed state and a second cross sectional area in the expanded state. The second cross-sectional area may be greater than the first cross-sectional area.

In certain embodiments, the expandable portion 210 of the second cannula 200 may be configured to transition between various states, such as, for example, the collapsed state and the expanded state, as well as various intermediate states between the collapsed and expanded states, such as partially expanded states. The collapsed state may have a first diameter and a first cross-sectional area. The expanded state may have a second diameter and a second cross-sectional area. The second diameter may be greater than the first diameter, and the second cross-sectional area may be greater than the first cross-sectional area. In certain embodiments the expandable portion is between 7 French (“Fr”) and 10 Fr when in the collapsed state. In some embodiments the expandable portion is between 20 Fr and 24 Fr in the expanded state. In some embodiments the expandable portion has minimal wall thickness. In certain embodiments the average thickness of the expandable portion's walls is between 0.0025 inches and 0.02 inches. The collapsed state may have a first length. The expanded state may have a second length. In some embodiments the second length is substantially the same as the first length (i.e., the expandable portion does not elongate or shorten during expansion). In certain embodiments the first length is between 10 cm and 75 cm. In certain embodiments the second length is between 10 cm and 75 cm. In other embodiments the second length is appreciably different from the first length (i.e., the expandable portion elongates or shortens during expansion).

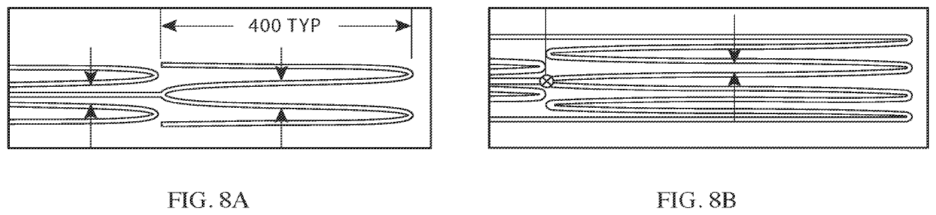

In some embodiments the expandable distal portion of the second cannula includes one or more materials and/or structures and/or components having properties allowing the expandable portion to reach full expansion at mean arterial pressure. For example, the expandable portion may include a metallic framework or structure, such as a stent. Various stents may be utilized by the device 10, such as those illustrated in FIG. 8A 8B, for example, parabolic stents, repeating wave stents, nitinol stents, metallic stents. In some examples, the framework may be coated or covered, partially or fully, with another material such as silicone or an elastomer, using a dip-coating or spin-coating technique. The metallic framework of a stent may define the largest possible diameter of the expandable portion in use.

In some embodiments the expandable portion includes materials that stretch (i.e., increase in one or more dimension) under load, such as, for example, knit, woven, and/or elastomeric tubing, fabrics, sheaths, coatings, and/or similar. Various elastomeric materials may be utilized by the device 10, such as, for example, tubes including silicone, and/or urethane. Various knit and/or woven materials may be utilized by the expandable portion, such as, for example, braided, wound, warp knit, and/or spun material, including, but not limited to, elastomers, fabrics, polyesters, and fibers. Various knit materials utilized by the expandable portion may be specifically configured to stretch in the presence of an internal pressure within a range of pressures and not stretch in the presence of internal pressures outside of the range. For example, in some embodiments the expandable portion includes a warp knit fabric formed of one or more fibers, including but not limited to mono-filament or multi-fillar line. Such warp knit fabric may be configured to permit movement of its individual fibers, in relation to themselves and/or others, such that the fabric expands when the expandable portion's internal pressure is equal to or greater than the fabric's minimum pressure. Such warp knit fabric may be configured to cause its individual fibers to be loaded and under tension when the expandable portion's internal pressures is equal to or greater than the fabric's maximum pressure, such that the warp knit fabric does not further expand and/or cause the expandable portion to further expand once the maximum pressure is reached. In some embodiments the expandable portion includes an elastomeric coating disposed over a base material. For example, various knit and/or woven materials utilized by the device 10 may be coated, such as, for example, dip-coated in an elastomer, and/or spin-coated in an elastomer. In some embodiments the expandable portion includes material that avoids, inhibits, or otherwise prevents endothelialization and/or tissue growth.

In some embodiments the expandable portion of the second cannula transitions from the collapsed state to the expanded state in response to a fluid flowing within it having sufficient pressure. In some embodiments the expandable portion is configured to expand in response to internal pressure being between 3 mmHg to 300 mmHg. In certain embodiments the expandable portion begins to transition (e.g., expands beyond the collapsed state to an intermediate state not equal to the expanded state) at a fluid flow pressure between 5 mmHg and 200 mmHg. In some embodiments the expanded state is reached at a predetermined pressure. In certain embodiments the predetermined pressure is between 7 mmHg and 30 mmHg. In other embodiment the predetermined pressure is between 30 mmHg and 60 mmHg. In other embodiments the predetermined pressure is between 60 mmHg and 160 mmHg. In other embodiments the predetermined pressure is between 160 mmHg and 300 mmHg.

In some embodiments, the expandable portion is configured to stretch beyond its initial dimensions when exposed to a great enough internal pressure and returns, at least substantially, to smaller dimensions or its initial dimensions when that internal pressure is removed or otherwise significantly reduced. In certain embodiments, the expandable portion may include elastic materials and/or knit materials capable of increasing in size when subject to sufficient pressures within the expandable portion.

In some embodiments the expandable portion is configured to be compressed to have reduced dimensions prior to placement and return to its initial dimensions when exposed to a sufficient internal pressure (e.g., 100 mmHg). In some embodiments, the expandable portion may include substantially non-stretchable materials, such as, for example, thermoplastics including, but not limited to, ePTFE, PTFE, FEP, and TPU. In some embodiments, the expandable portion may include pleats. In certain embodiments the expandable portion may include a thin material folded, twisted, spun, and/or crumpled from its initial dimensions to smaller dimensions prior to use. For example, the expandable portion may include a substantially non-stretchable material having pleats that is folded prior to insertion such that the expandable portion is placed in a compressed configuration of reduced dimensions. In such embodiments, the expandable portion may include one or more releasable retention mechanism, including, but not limited to, sutures, ties, band, sheaths, and coverings. A releasable retention mechanism may be configured to keep the expandable portion in the compressed configuration during insertion and may be configured to release from retaining the expandable portion once placed in a desired location such that the expandable portion is permitted to return to its initial dimensions in the presence of sufficient internal pressure.

In some embodiments the expandable portion is self-expanding. In certain embodiments the expandable portion may include structures or mechanisms that are able to be compressed and restrained until the expandable portion is placed in a desired location, at which time the structures or mechanism are unrestrained and automatically return to their uncompressed shape and/or size. For example, in one embodiment the expandable portion includes one or more stents that can be compressed and then restrained by a sheath, which when removed permits the stents to expand to their original dimensions.

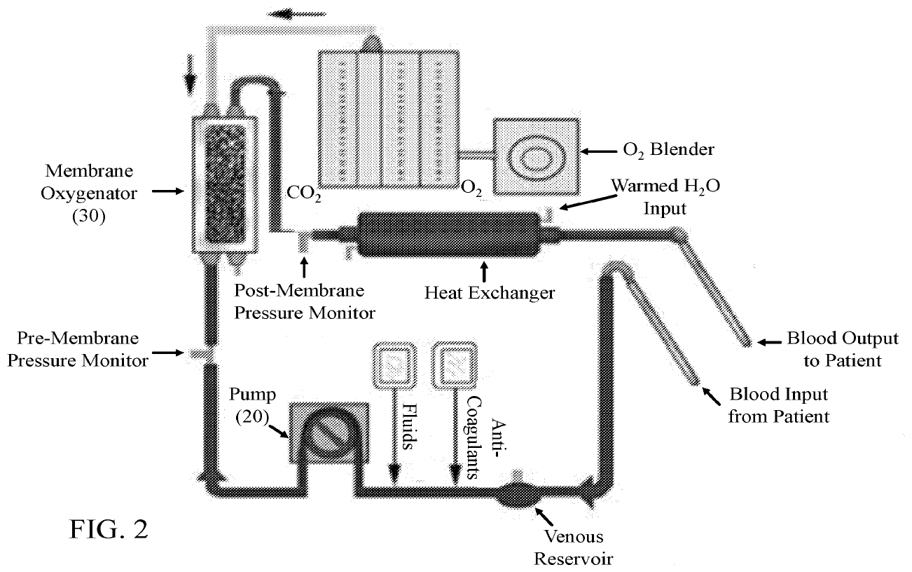

Referring again to FIG. 3, in some embodiments the first cannula includes one or more perforations 110. In some embodiments the first cannula 100 may include a port section 120. The first port section 120 may be configured for connecting the first cannula 100 to ECMO machinery, such as the ECMO machinery shown in FIG. 2. For example, the first port section 120 may secure the first cannula 100 as part of an outflow fluidic pathway establishing fluidic communication between venous vasculature of a patient and components of the ECMO system. Similarly, the second cannula 200 may include a port section 220. The second port section 220 may be configured for connecting the second cannula to the ECMO machinery, such as the ECMO machinery shown in FIG. 2. For example, the second port section 220 may secure the second cannula 200 as part of an inflow fluidic pathway establishing fluidic communication between the ECMO machinery with part of the patient's vasculature and/or cardiac anatomy.

In some embodiments, the first cannula 100 may include a connector section 140 configured to operatively couple or connect the second cannula 200 to the first cannula 100. In certain embodiments the connector section 140 is configured to sealably permit the second cannula 200 to translate within the first cannula 100. In some embodiments, the first cannula 100 includes an aperture 130 where the second cannula 200 may enter and/or exit the first cannula 100. In further embodiments the aperture 130 is configured to sealably allow the second cannula 200 to enter exit the first cannula. In some embodiments the aperture 130 is configured to permit the second cannula 200 to move within the first cannula 100. In certain embodiments the second cannula 200 includes a coupling 230 for connecting the expandable distal portion 210 to the remainder of the second cannula 200.

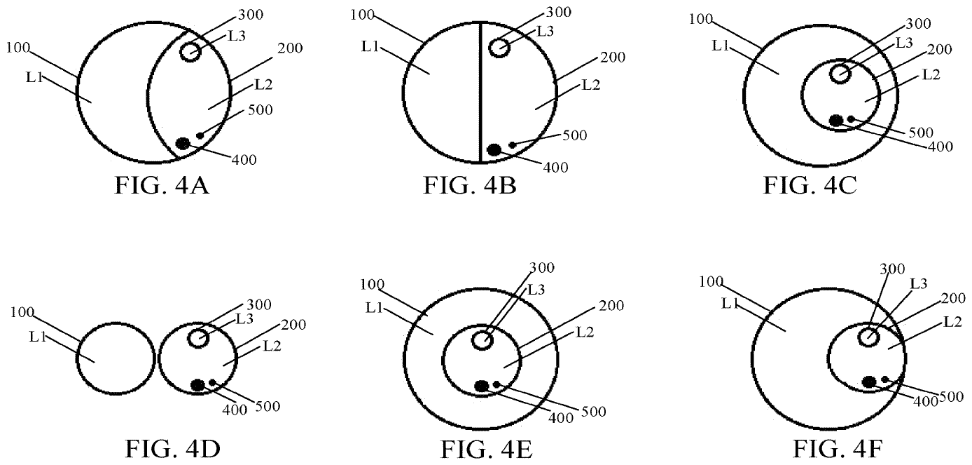

The first cannula 100 and second cannula 200 may be in various positions relative to each other. Referring to FIGS. 4A through 4F, exemplary cross-sectional views of a vascular device having a first cannula 100 and a second cannula 200 are depicted. In some embodiments the first and second cannulas are lateral to each other (e.g., FIGS. 4A, 4B, 4D). For example, the two cannulas may be side-by side, and one cannula may form a crescent cross-section while the other cannula forms a complementary cross-section (e.g., FIG. 4A). The two cannulas may both have semi-circular cross-sections (e.g., FIG. 4B). The two cannulas may be parallel to each other, and both have circular cross sections (e.g., FIG. 4D). In some embodiments a portion of the second cannula 200 may be disposed partially or entirely within the first cannula 100 (e.g., FIGS. 3, 4C, 4E, 4F). For example, the two cannulas may have circular cross-sections, with the second cannula 200 having a smaller diameter than the first cannula 100 and being entirely within the other (e.g., FIGS. 4C, 4E, 4F). The two cannulas may be concentrically aligned (e.g., FIG. 4E) or non-concentrically aligned (e.g., FIG. 4C). The cannula having the smaller diameter may touch, connect to, or otherwise be aligned along the interior surface of the cannula having the larger diameter (e.g., FIG. 4F). In some embodiments a portion of the second cannula is parallel to the first cannula and another portion of the second cannula is non-parallel to the first cannula. For example, a portion of the second cannula may be partially within the first cannula (e.g., the embodiment shown in FIG. 3). In some embodiments various portions of the first cannula are in different positions relative to each other. For example, one portion of the first cannula may be non-parallel to another portion of the first cannula (e.g., the embodiment shown in FIG. 3). In some embodiments various portions of the second cannula are in different positions relative to each other. For example, one portion of the second cannula may be non-parallel to another portion of the second cannula (e.g., the embodiment shown in FIG. 3).

Referring again to FIG. 3, in some embodiments the device 10 includes a guide 400 that is disposed at least partially within, or translatable with respect to, the first cannula 100, the second cannula 200 or both. The guide 400 may be a wire, push catheter, or other component configured for maneuvering the expandable distal portion 210 into various positions. The guide 400 may sealably connect to the second cannula 200 via a connector section 240. The expandable portion 210 may be configured to be back loaded over the guide 400 and may be connected to the guide 400 at the distal end of the expandable portion 210. In certain embodiments, the guide 400 is releasably connected to the expandable portion 210, and once fluid has started flowing through the second cannula 200 and the expandable portion 210 is in the expanded state, the guide 400 may be released and removed from the expandable section 210 without moving the expandable section 210 from its location within the patient's vasculature. Connector section 240 may enable the guide 400 to be manipulated to maneuver the expandable portion 210 while keeping the connection sealed and may enable guide 400 to be removed from the expandable portion 210 without moving the device 10.

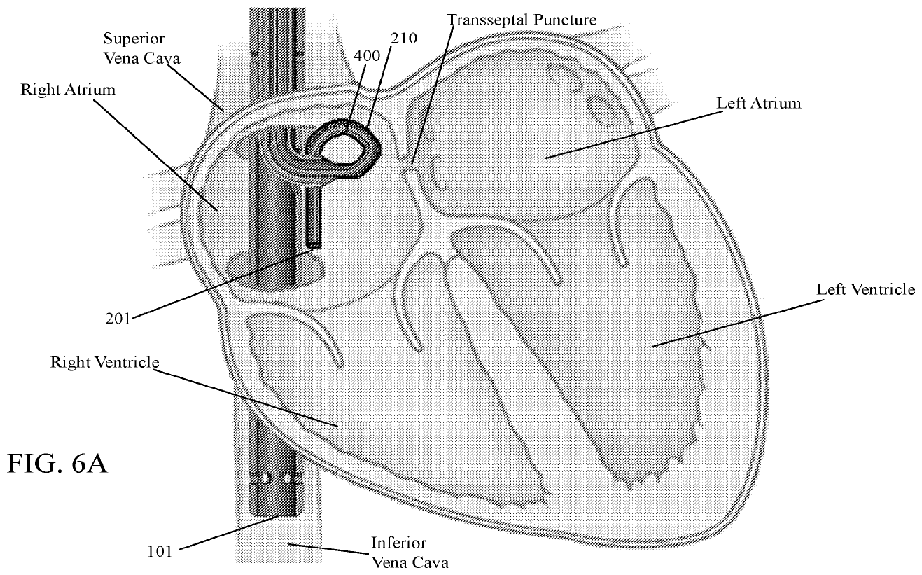

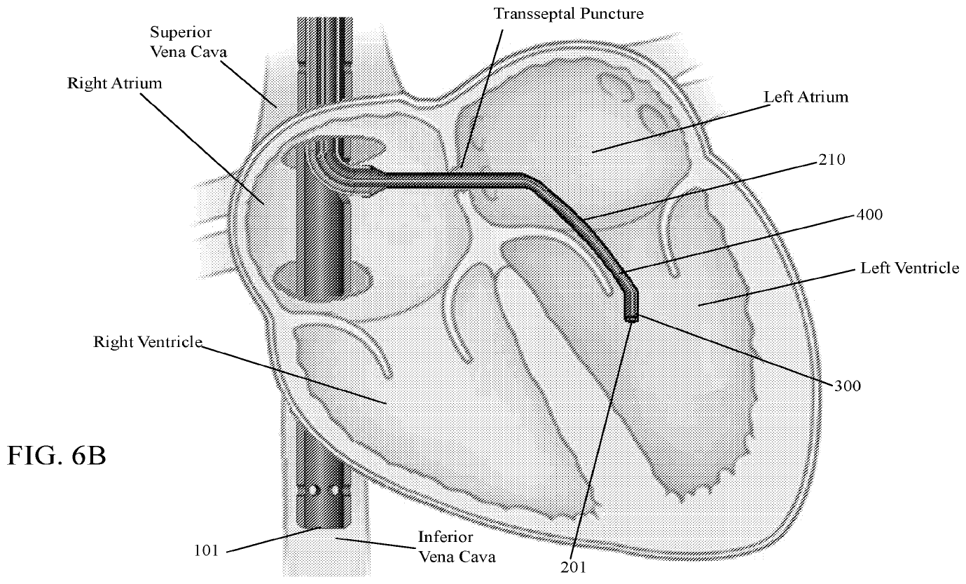

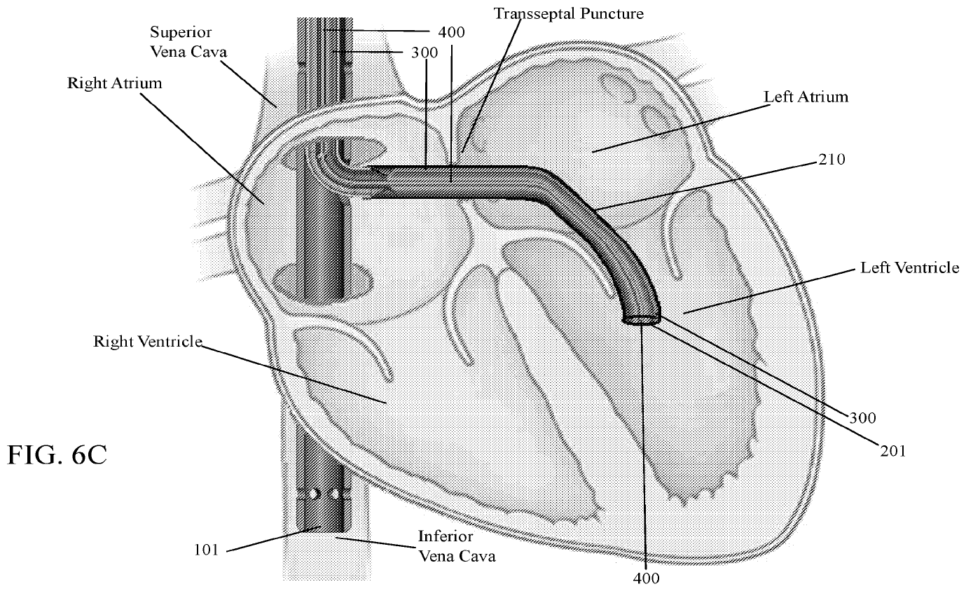

In some embodiments the device 10 includes a third cannula 300, having a proximal opening, a distal opening, and defining a third lumen L3. The third cannula 300 may be in various positions relative to first cannula 100 and the second cannula 200. In some embodiments the third cannula 300 extends from its proximal opening within the first cannula 100, into the second cannula 200, continuing to its distal opening external to the second cannula 200. In certain embodiments the third cannula 300 is configured to allow drainage of an area of the patient's vasculature that is near the distal opening 201 of the second cannula, such as, for example, the patient's Left Ventricle as illustrated in FIG. 6C. The third cannula 300 may be flexible and/or expandable.

In some embodiments, the device 10 includes a third cannula 300 and a guide 400. The guide 400 may be configured to be removed from, such as, for example, pulled out of, the second cannula 200 after the expandable section 210 is in the expanded state. The device 10 may be configured for the guide 400 to then be inserted, such as, for example, pushed, into the third cannula 300. The guide 400 may be configured to maintain patency of the third cannula 300 as it acts to drain an area of the patient's vasculature or cardiac anatomy.

In some embodiments device 10 includes wiring 500. The wiring may be configured to couple to one or more sensors and/or one or more heating elements. Various sensors may be within or external to the first cannula 100, the second cannula 200, and/or a third cannula 300. Some embodiments include sensors for monitoring conditions within the device or within the patient, such as, but not limited to, for example, sensors configured for monitoring pressure (for example, pressure within the first cannula, second cannula, third cannula, a vein, an artery, or cardiac area), flow rate, oxygenation, temperature, thermodilution, and/or electrical signals. In some embodiments the device 10 includes a wiring connection for providing the wiring 500 with access to a cannula of the device.

As would be appreciated by one skilled in the art, FIGS. 1, 3, 5A-5C and 6A-6C are for exemplary purposes only and the first cannula 100, second cannula 200, third cannula 300, and the expandable distal portion 210 may include any combination of shapes and sizes to form any combination of sized and shaped cannulas, and the device 10, as well as the components thereof, may be constructed from any combination of materials using any combination of methods known in the art, depending of the desired application.

Referring to FIG. 1, in operation of some embodiments, the vascular device 10 is configured for use within an ECMO system 1. The device 10 may be connected to external components (i.e., not in-vivo) of the ECMO system, such as a pump 20 and an oxygenator 30. The proximal opening of the first cannula 100 may be connected to a first external cannula to establish a first fluidic pathway between the distal opening 101 of the first cannula 100 and the pump 20. The proximal opening of the second cannula 200 may be connected to a second external cannula to establish a second fluidic pathway between the oxygenator 30 and the distal opening 201 of the second cannula 200. The Pump 20 may be in fluid communication with the oxygenator 20. External components of the ECMO system, for example ECMO machinery, such as shown in FIG. 2, may include various components, such as, but not limited to, oxygenators (e.g., membrane oxygenator 30), pumps (e.g., pump 20), pressure monitors, heat exchangers, O₂ blenders, reservoirs, catheters, cannulas, connectors, supply of H₂O, inputs for warmed H₂O, supply of anti-coagulants, inputs for anti-coagulants, supply of other fluids, and inputs for other fluids.

The device 10 may be inserted into a patient's vasculature and advanced to a first target position. Referring to FIGS. 1, 5A and 6A, an illustrative use of a vascular medical device and ECMO system of the present disclosure follows. The device 10 may be inserted into a patient via the patient's Jugular Vein and advanced into the patient's Vena Cava such that it is placed in a first target position. For example, the first target position may be where the first cannula 100 extends from its distal opening 101 within the patient's Inferior Vena Cava, traveling into the patient's Right Atrium and continuing out of the patient's Right Atrium into the patient's Superior Vena Cava until it exits the patient's Jugular Vein and connects to the respective external cannula of the ECMO system 1. In that first target position, the expandable distal portion 210 and the distal opening 201 of the second cannula 200 are within the patient's Right Atrium. When first inserted, the expandable distal portion 210 is preferably in a collapsed state. While in the collapsed state, the expandable portion 210 may be maneuvered to place the device 10 in a second target position where the distal opening 201 of the second cannula 200 is in a second location within the patient's vasculature or cardiac anatomy. In some embodiments, a guide wire 400 may be manipulated to control the movement of the expandable portion 210 and thereby placement of the second cannula's distal opening 201.

Referring to FIG. 1, once connected to external components of an ECMO system and inserted into the patient's vasculature, the first cannula 100 preferably forms part of an outflow fluidic pathway establishing fluidic communication between the patient's venous vasculature and a pump 20 of the ECMO system, and the second cannula 200 preferably forms part of an inflow fluidic pathway establishing fluidic communication between an oxygenator 30 and part of the patient's vasculature or cardiac anatomy.

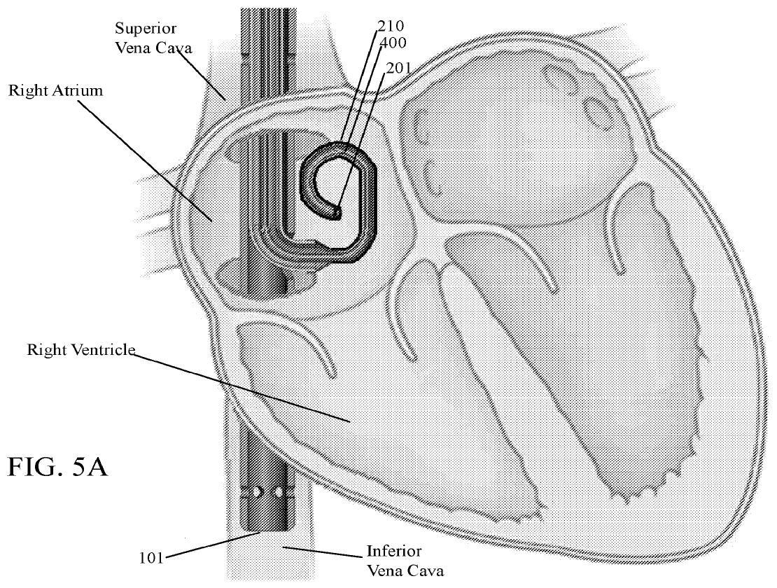

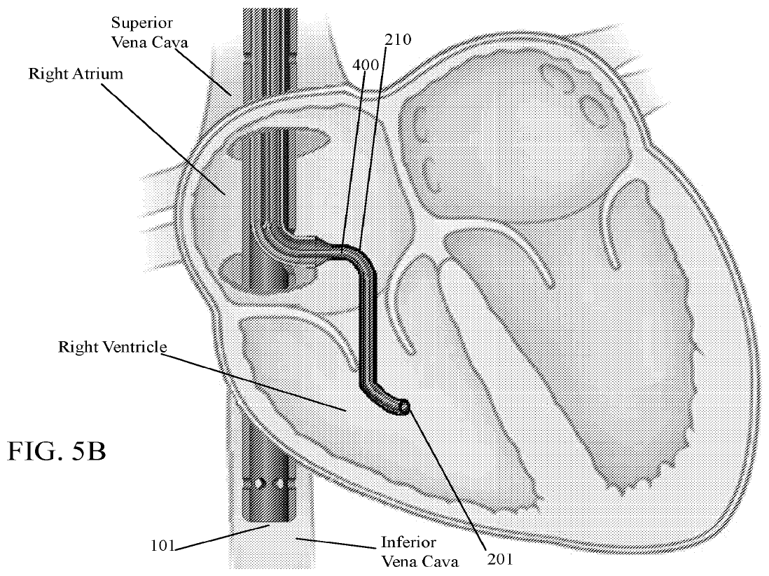

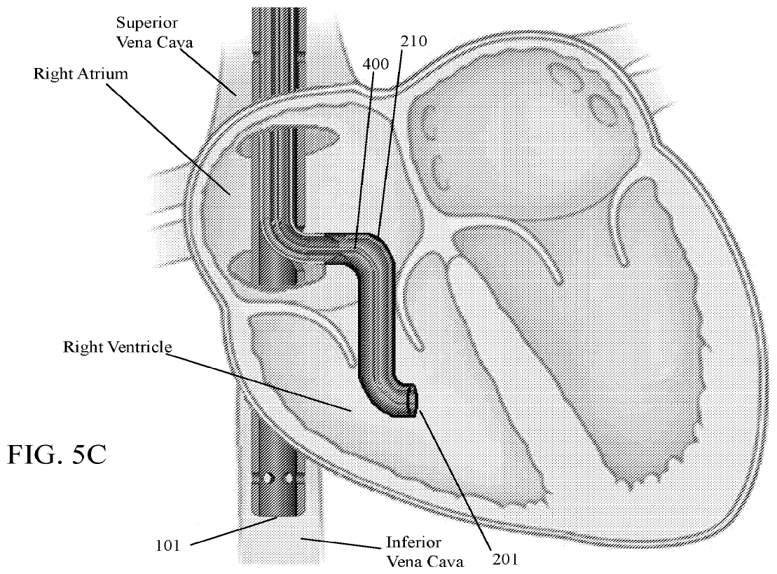

Referring to FIGS. 5B and 6B, the device 10 is shown after being maneuvered and placed in various second target positions. In FIG. 5B the expandable portion 210 has been maneuvered through the patient's Tricuspid Valve to place the second cannula's distal opening 201 in the patient's Right Ventricle. In FIG. 6B the expandable portion 210 has been maneuvered through a Transseptal Puncture, into the patient's Left Atrium and through the patient's Mitral Valve to place the second cannula's distal opening 201 in the patient's Left Ventricle.

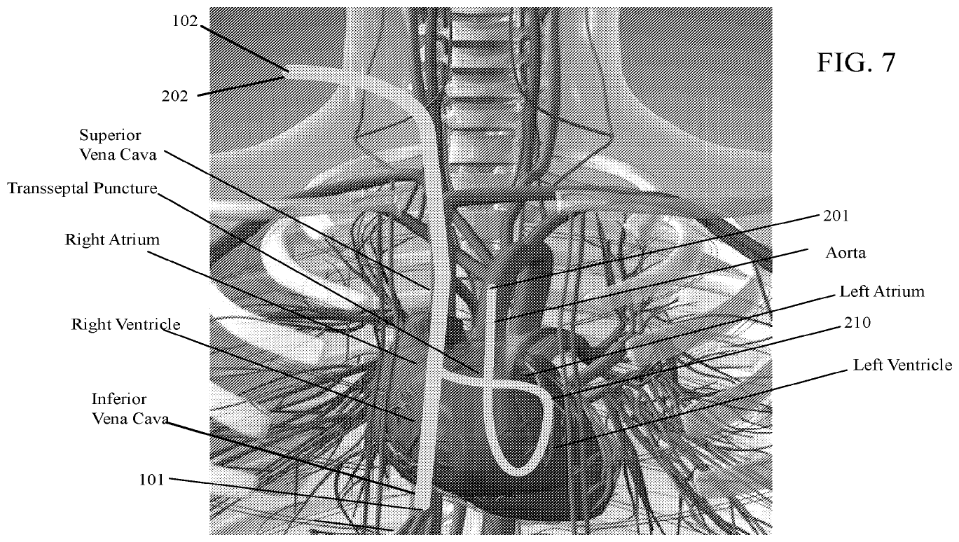

Referring to FIGS. 5C, 6C, and 7, once placed in the second target position, the expandable distal portion 210 is preferably transitioned from the collapsed state to an expanded state. With the distal portion 210 in the second target position, the ECMO system 1 can pump, via the pump 20, the patient's deoxygenated blood out of the patient's venous vasculature and/or right atrium, via the first cannula 100, to the oxygenator 30 and return oxygenated blood via the second cannula 200 to the second target position within the patient's arterial vasculature and/or cardiac anatomy. Transitioning to the expanded state of the expandable distal portion 210 may result from increasing the pressure within the expandable portion 210, such as, for example, by pumping the returned oxygenated blood at sufficient flow rates. For example, referring to FIGS. 5C and 6C, the returning oxygenated blood can be pumped through the second cannula 200 at a rate having pressure great enough to increase the dimensions of the expandable distal portion 210 of the second cannula 200, thereby placing the expandable distal portion 210 in the expanded state having dimensions sufficient for oxygenated blood to exit the distal end 201 of the expandable portion 210 and flow into the patient's Right Ventricle (as shown in FIG. 4C) or Left Ventricle (as shown in FIG. 5C) or Aorta (FIG. 7), at rates sufficient to support (i.e., supplement or maintain) the patient's circulation.

In some embodiments, the device may include a third cannula 300 which may be utilized as a drain line for the area of the patient's vasculature or cardiac anatomy where the distal opening 201 of the second cannula 200 is located when oxygenated blood is pumped back into the patient via the second cannula 200. In certain embodiments, the third cannula establishes fluidic communication between the patient's vasculature or cardiac anatomy and the first cannula 100. In further embodiments, a guide, such as a guide wire, is configured to release and be withdrawn from the expandable portion of the second cannula once the distal end of the second cannula is placed in the second location, and then the guide may be advanced into the third cannula where the guide may act to maintain patency of the third cannula while it functions as a drain line. In some embodiments the expandable portion may define multiple lumens and may include at least a portion of the third cannula. For example, the expandable portion may include a main lumen configured to accommodate the return flow of oxygenated blood and a secondary lumen that may include at least a portion of the third cannula configured to act as a drain line.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

What is claimed is:

1. A vascular medical device comprising:
a first cannula having a proximal opening and a distal opening, and defining a first lumen; and
a second cannula having a proximal opening and a distal opening, and defining a second lumen, the second cannula comprising an expandable distal portion configured to transition between a collapsed state having a first cross-sectional area, to an expanded state having a second cross-sectional area that is greater than the first cross-sectional area.

2. The device according to claim 1, wherein the expandable distal portion comprises a stent.

3. The device according to claim 1, wherein the expandable distal portion comprises an elastomeric tube.

4. The device according to claim 1, wherein the expandable distal portion comprises a knit material.

5. The device according to claim 1, wherein the expandable distal portion comprises an elastomeric coating.

6. The device according to claim 1, wherein the expandable distal portion is configured and arranged to transition in response to a predetermined pressure within the first lumen.

7. The device according to claim 6, wherein the predetermined pressure is between 7-30 mm Hg.

8. The device according to claim 6, wherein the predetermined pressure is between 60 -160 mm Hg.

9. The device according to claim 1, wherein the expandable distal portion is capable of percutaneous delivery in the collapsed state.

10. The device according to claim 9, wherein the collapsed state is between 7 and 10 French.

11. The device according to claim 1, wherein the expanded state is between 20 and 24 French.

12. The device according to claim 1, wherein the first cannula and the second cannula are at least partially concentric.

13. The device according to claim 1, wherein the first cannula is lateral to the second cannula.

14. The device according to claim 1, wherein the second cannula is at least partially disposed outside the first cannula.

15. The device according to claim 1, further comprising a guidewire configured to controllably maneuver the expandable distal portion of the second cannula.

16. A method of treatment comprising:
providing a first cannula having a proximal opening and a distal opening, and defining a first lumen;
providing a second cannula having a proximal opening and a distal opening, and defining a second lumen, the second cannula comprising an expandable distal portion;
placing the second cannula at a first target position;
placing the expandable distal portion of the second cannula at a second target position; and
transitioning the expandable distal portion from a collapsed state having a first cross-sectional area, to an expanded state having a second cross-sectional area that is greater than the first cross-sectional area.

17. The method of claim 16 wherein placing the second cannula at a first target position comprises percutaneously delivering the second cannula in the collapsed state.

18. The method of claim 16 further comprising providing a guidewire configured to controllably maneuver the expandable distal portion of the second cannula, wherein placing the expandable distal portion at the second target position comprises using the guidewire to maneuver the second cannula to the second target position.

19. An extracorporeal membrane oxygenator system for extracorporeal oxygenation of blood and removal of carbon dioxide from said blood, the system comprising:
a pump for pumping blood;
an oxygenator for exchanging oxygen and carbon dioxide within the pumped blood; and
a vascular medical device comprising:
a first cannula having a proximal opening and a distal opening, and defining a first lumen; and
a second cannula having a proximal opening and a distal opening, and defining a second lumen, the second cannula comprising an expandable distal portion configured to transition between a collapsed state having a first cross-sectional area, to an expanded state having a second cross-sectional area that is greater than the first cross-sectional area.

20. The system of claim 19, wherein:
the distal opening of the first cannula is configured to be placed within a vein of a patient;
the proximal opening of the first cannula is configured to be placed in fluid communication with the pump;
the pump is configured to placed in fluid communication with the oxygenator;
the oxygenator is configured to placed in fluid communication with the proximal opening of the second cannula; and
the distal opening of the second cannula is configured to be placed within a chamber of the patient's heart.
